(19) United States
(12) Patent Application Publication
(10) Pub. No.: US 2019/0254644 A1
(43) Pub. Date: Aug. 22, 2019

(54) BIOPSY FORCEPS WITH CAM MECHANISM

(71) Applicant: BOSTON SCIENTIFIC LIMITED, Hamilton (BM)

(72) Inventors: Agrim Mishra, New Delhi (IN); Nishant Randhawa, S.A.S. Nagar (IN); Hitendra Purohit, Vadodara (IN); Deepak Kumar Sharma, Muzaffarnafar (IN)

(21) Appl. No.: 16/253,951

(22) Filed: Jan. 22, 2019

Related U.S. Application Data
(60) Provisional application No. 62/633,856, filed on Feb. 22, 2018.

(51) Int. Cl. A61B 10/06; A61B 10/04; A61B 17/295

(52) U.S. Cl. CPC A61B 10/06; A61B 2017/2936; A61B 17/295; A61B 10/04

(57) ABSTRACT

A biopsy forceps device includes a tension member and an end effector which includes jaws movable between an open configuration, in which the jaws are separated from one another to receive target tissue therebetween, and a closed configuration, in which cutting edges of the jaws are moved toward one another to cut a portion of the target tissue from surrounding tissue. The jaws defines a tissue receiving space therebetween to house the cut tissue. The jaws are pivotable relative to one another. The end effector further includes a tension member attachment coupled to the tension member; a distal end of which terminates at a sharp spike configured to penetrate the tissue. The attachment is movably coupled to the jaws so that distal movement of the attachment moves the jaws to the open configuration, while proximal movement thereof moves the jaws to the closed configuration.

### PRIORITY CLAIM

The present disclosure claims priority to U.S. Provisional Patent Application Ser. No. 62/633,856 filed Feb. 22, 2018; the disclosure of which is incorporated herewith by reference.

### FIELD

The present disclosure relates to endoscopic instruments, and more specifically, to biopsy forceps for use in endoscopic procedures.

### BACKGROUND

Tissue samples are often examined to determine the presence of a pathological disorder. Endoscopic biopsy forceps may be used in conjunction with an endoscope for taking certain tissue samples from the human body for analysis. Often, the samples must be obtained from deep within the body at a location that is difficult to access by simply using forceps jaws (i.e., tissue from an area accessible via a tortuous biliary path). In certain cases, the quality of tissue that is easily accessible by a physician may not be satisfactory for pathologists to make an accurate diagnosis. Furthermore, forceps jaws are often difficult to maneuver for tangential bites.

### SUMMARY

The present disclosure is directed to a biopsy forceps device comprising a tension member extending from a proximal end to a distal end and an end effector including first and second jaws movable between an open configuration, in which the jaws are separated from one another to receive target tissue therebetween , and a closed configuration, in which cutting edges of the jaws are moved toward one another to cut a portion of the target tissue from surrounding tissue, the first and second jaws defining a tissue receiving space therebetween to house the cut tissue, the first and second jaws being pivotable relative to one another, the end effector further including a tension member attachment extending from a distal end to a proximal end coupled to the tension member, the distal end of the tension member attachment terminating at a sharp spike configured to penetrate the tissue, the tension member attachment being movably coupled to the first and second jaws so that distal movement of the tension member attachment moves the jaws to the open configuration while proximal movement thereof moves the jaws to the closed configuration.

In an embodiment, the first and second jaws include concave inner surfaces defining a substantially hemispherical cup.

In an embodiment, the tension member attachment includes a proximal part and a distal part, the distal part including first and second cam slots, the first and second cam slots being positioned on opposing outer surfaces of the tension member attachment.

In an embodiment, the first and second jaws each include a cam pin, the cam pin of the first jaw being slidably received within the first cam slot while the cam pin of the second jaw is slidably received within the second cam slot so that movement of tension member attachment relative to the cam pins of the first and second jaws pivots the first and second jaw in opposing directions.

In an embodiment, the first and second cam slots are substantially arcuate in shape, the first and second cam slots curving in opposite directions and crossing one another with a medial portion of each cam slot overlapping.

In an embodiment, the proximal part of the tension member attachment includes a blind hole open at a proximal end in which a distal end of the control wire is received.

In an embodiment, the end effector has a length of less than 5 mm.

In an embodiment, the end effector has a length of less than approximately 4.95 mm.

In an embodiment, the device further includes a clevis, the clevis including first and second arms extending distally from a proximal portion, the proximal portion including a central lumen sized and shaped to receive the tension member attachment therethrough, the arms defining a jaw receiving space therebetween for receiving a proximal portion of each of the first and second arms, the first jaw being pivotably coupled to the first arm and the second jaw being pivotably coupled to the second arm.

The present disclosure is also directed to biopsy forceps system for sampling tissue comprising a proximal assembly including an actuator, a tension member extending from a distal end to a proximal end coupled to the proximal assembly and a distal assembly including first and second jaws movable between an open configuration, in which the jaws are separated from one another to receive target tissue therebetween, and a closed configuration, in which cutting edges of the jaws are moved toward one another to cut the target tissue from surrounding tissue, the first and second jaws defining a tissue receiving space therebetween to house the cut tissue, the first and second jaws being pivotable relative to one another, the distal assembly further including a tension member attachment extending from a distal end to a proximal end coupled to the tension member, the distal end of the tension member attachment terminating at a tissue penetrating spike, the tension member attachment movably coupled to the first and second jaws so that distal movement of the tension member attachment pivots the jaws to the open configuration while proximal movement of the tension member attachment pivots the jaws to the closed configuration wherein actuation of the proximal assembly causes the tension member attachment to move proximally and distally relative to the first and second jaws.

In an embodiment, the actuator comprises a handle and a spool, the spool being coupled to the control wire and slidable along a longitudinal axis of the handle.

In an embodiment, the tension member attachment includes a proximal part and a distal part, the distal part including first and second cam slots, the first and second cam slots being positioned on opposing outer surfaces of the tension member attachment.

In an embodiment, each of the first and second jaws includes a cam pin, the cam pin of the first jaw being slidably received in the first cam slot while the cam pin of the second jaw is slidably received the second cam slot so that movement of the tension member attachment relative to the first and second jaws pivots the first and second jaws in opposing directions.

In an embodiment, the first and second cam slots are arcuate, curve in opposite directions and cross one another.

In an embodiment, the system further includes a clevis, the clevis including first and second arms extending distally from a proximal portion of the clevis, the proximal portion including a central lumen sized and shaped to receive the tension wire attachment therein, each of the arms defining a jaw receiving space therebetween for receiving a proximal portion of a corresponding one of the first and second arms, the first jaw being pivotably coupled to the first arm via and the second jaw being pivotably coupled to the second arm.

The present disclosure is also directed to a method of obtaining a tissue sample comprising inserting a distal portion of a biopsy forceps assembly to a target area within a living body, the distal portion including a tension member extending from a proximal end to a distal end and an end effector including first and second jaws movable between an open configuration in which the first and second jaws are separated from one another too receive target tissue therebetween, and a closed configuration, in which cutting edges of the first and second jaws are moved toward one another to cut the target tissue away from surrounding tissue, the first and second jaws defining a tissue receiving space therebetween to house the cut tissue, moving the tension member distally relative to the first and second jaws to move a tension member attachment coupled to the tension member distally so that a spike on a distal end of the tension member attachment penetrates the target tissue, the tension member attachment being movably coupled to the first and second jaws so that distal movement of the tension member attachment relative to the first and second jaws pivots the first and second jaws to the open configuration, and moving the tension member proximally relative to the first and second jaws to pivot the jaws to the closed configuration so that the cutting edges of the first and second jaws sever the target tissue received therebetween from the surrounding tissue.

In an embodiment, the method further includes inserting the biopsy forceps assembly through the working channel of an endoscope.

In an embodiment, the tension member attachment includes a proximal part and a distal part, the distal part including first and second cam slots, the first and second cam slots being positioned on opposing outer surfaces of the core wire attachment.

In an embodiment, the first and second jaws each include a cam pin, the cam pin of the first jaw being sized and shaped to slidably move within the first cam slot to pivot the first jaw in a first direction while the cam pin of the second jaw is configured slidably move within the second cam slot to pivot the second jaw in a second, opposing direction.

In an embodiment, the method further comprises actuating the distal portion via a proximal assembly coupled to the proximal end of the tension member.

### BRIEF DESCRIPTION

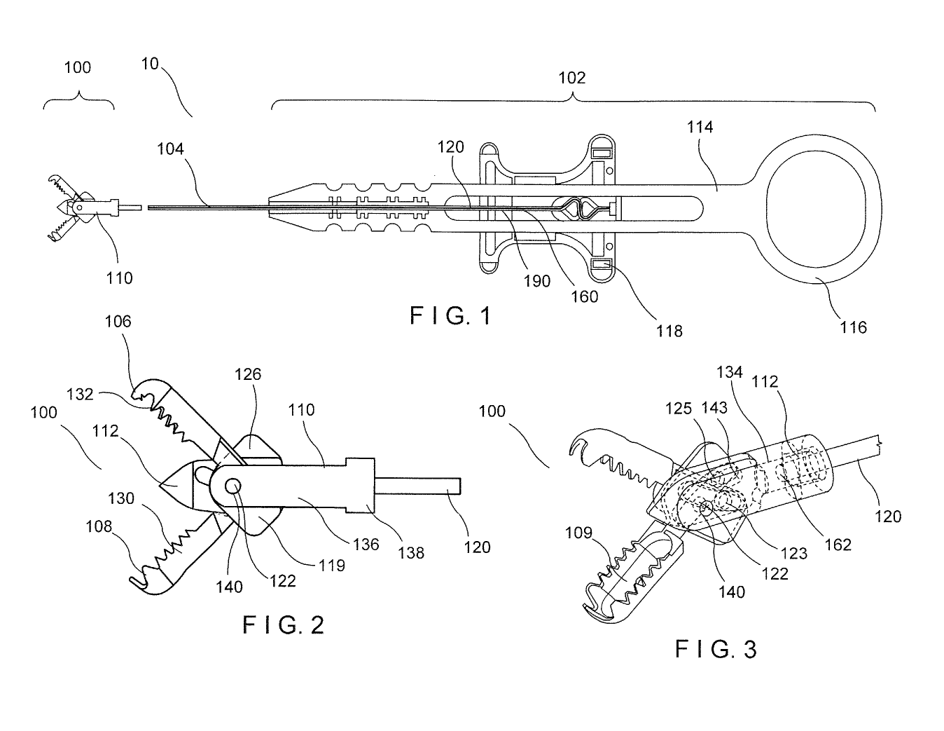

FIG. 1 shows a perspective view of a forceps assembly according to an exemplary embodiment of the present disclosure;

FIG. 2 shows a perspective view of a distal end effector assembly of the forceps assembly of FIG. 1 according to an exemplary embodiment of the present disclosure;

FIG. 3 shows a perspective, partially transparent view of the distal end effector assembly of FIG. 2;

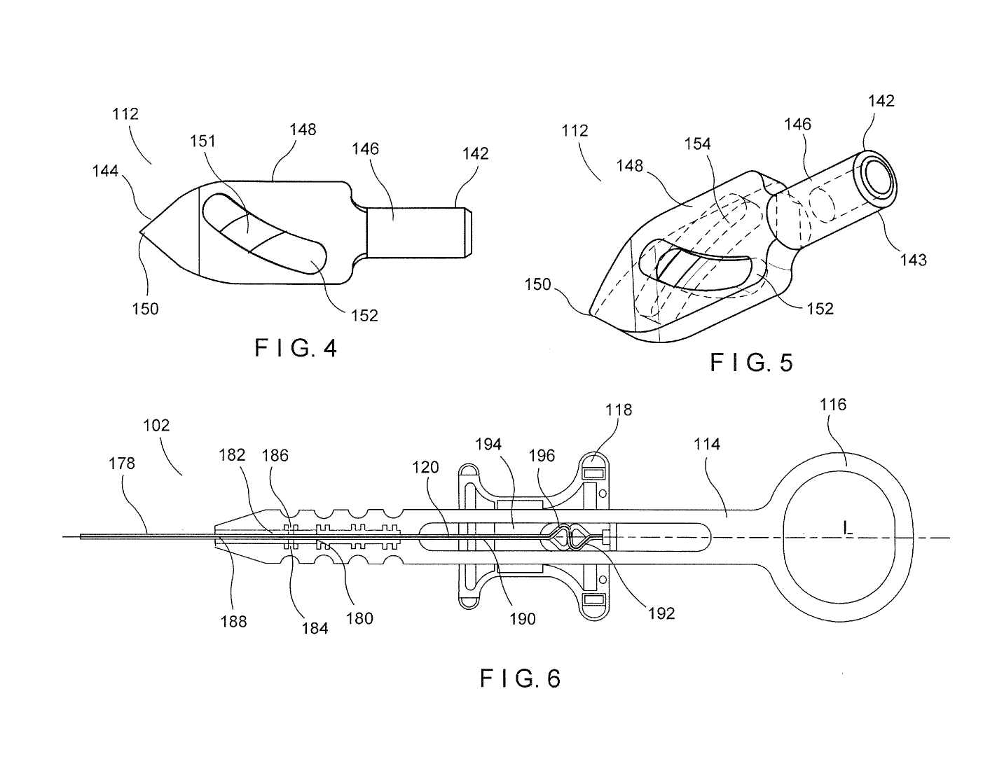

FIG. 4 shows a side view of a core wire attachment of the forceps assembly of FIG. 1 according to an exemplary embodiment of the present disclosure;

FIG. 5 shows a perspective, transparent view of the core wire attachment of FIG. 4;

FIG. 6 shows a side, partially transparent view of a proximal assembly of the forceps assembly of FIG. 1 according to an exemplary embodiment of the present disclosure;

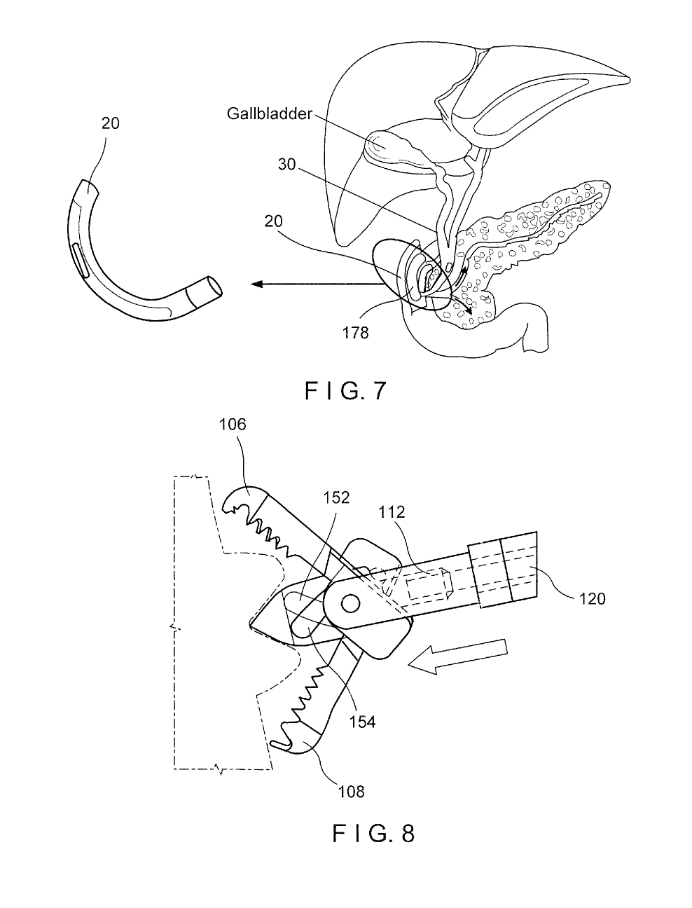

FIG. 7 shows a perspective view of a method of use of the forceps assembly of FIG. 1;

FIG. 8 shows a perspective view of a method of use of the forceps assembly of FIG. 1;

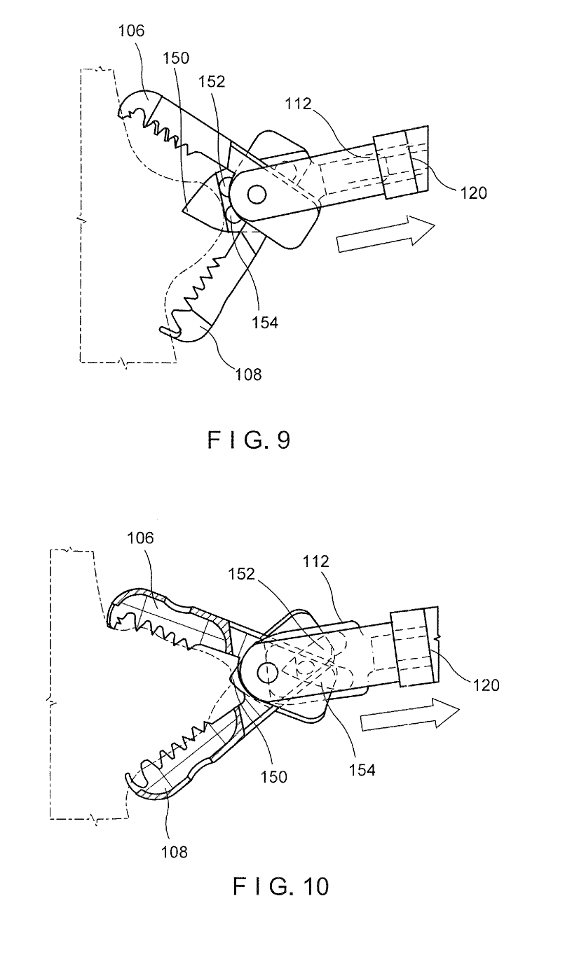

FIG. 9 shows a perspective view of a method of use of the forceps assembly of FIG. 1;

FIG. 10 shows a perspective view of a method of use of the forceps assembly of FIG. 1;

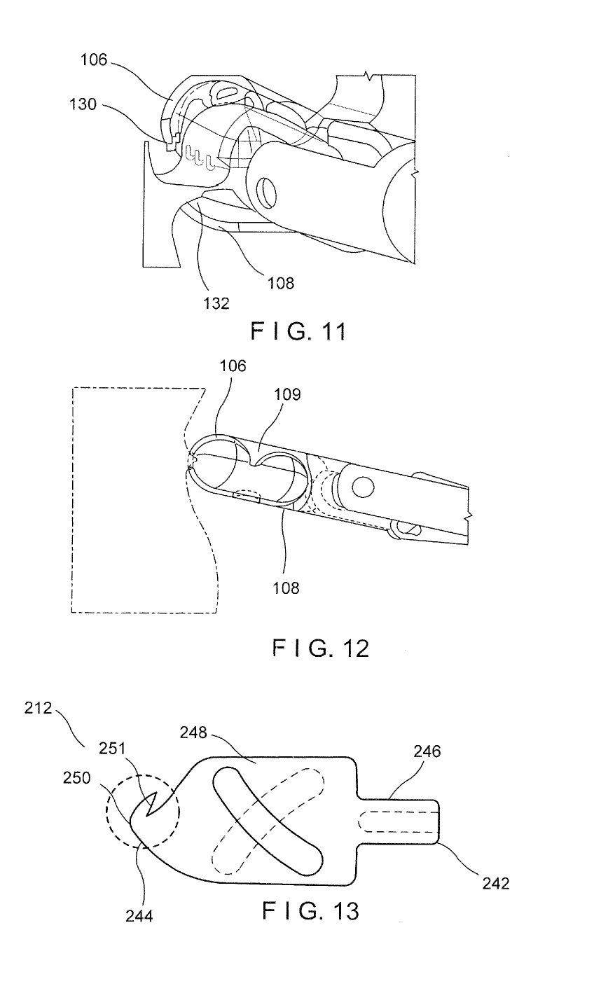

FIG. 11 shows a perspective view of a method of use of the forceps assembly of FIG. 1;

FIG. 12 shows a perspective view of a method of use of the forceps assembly of FIG. 1;

FIG. 13 shows a side, partially transparent view of a core wire attachment of the forceps assembly of FIG. 1 according to another exemplary embodiment of the present disclosure;

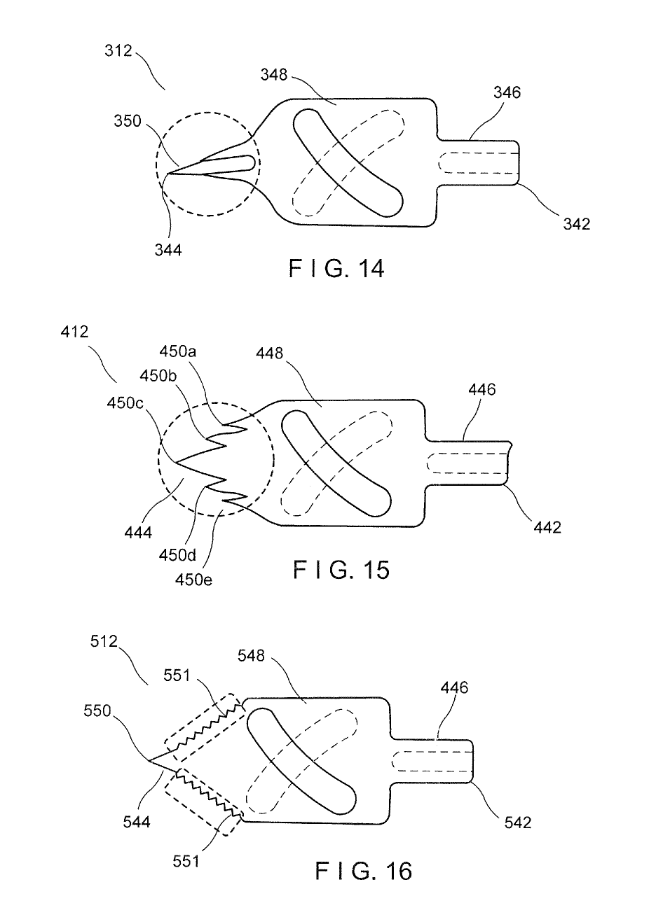

FIG. 14 shows a side, partially transparent view of a core wire attachment of the forceps assembly of FIG. 1 according to a third exemplary embodiment of the present disclosure;

FIG. 15 shows a side, partially transparent view of a core wire attachment of the forceps assembly of FIG. 1 according to a fourth exemplary embodiment of the present disclosure; and

FIG. 16 shows a side, partially transparent view of a core wire attachment of the forceps assembly of FIG. 1 according to a fifth exemplary embodiment of the present disclosure.

### DETAILED DESCRIPTION

The present disclosure may be further understood with reference to the following description and the appended drawings, wherein like elements are referred to with the same reference numerals. The present disclosure relates to an endoscopic forceps assembly for severing and retaining tissue samples. Exemplary embodiments of the present disclosure describe a forceps assembly that can be advanced through a working channel of an endoscope, including, for example, a SpyScope™, or any other endoscopic device specifically designed and/or sized for use with the forceps assembly, and into a tissue tract. Current embodiments also include a more compact forceps design for increasing the passability and maneuverability of the forceps assembly through tight curvatures within the working channels of the endoscopic devices as well as through a tortuous lumen of a living body. It should be noted that the terms “proximal” and “distal,” as used herein, are intended to refer to toward (proximal) and away from (distal) a user of the device.

A forceps assembly, according to an exemplary embodiment of the present disclosure, is depicted in FIG. 1. The forceps assembly 10 includes a distal end effector assembly 100, a proximal actuator assembly 102, and an elongate member 104 connecting the distal assembly 100 to the proximal actuator 102. The distal assembly 100, as shown in FIG. 2, includes a first jaw 106, a second jaw 108, a clevis 110 and a core wire attachment 112. The proximal assembly 102, as shown in FIGS. 1 and 6, includes a handle 114 including a proximal thumb ring 116, and a spool 118 that slides relative to the handle 114. The elongate member 104, in the present embodiment, is a coiled member and houses a tension member such as a control wire 120 that extends from the proximal assembly 102 to the distal assembly 100.

FIGS. 2-3 depict the distal assembly 100 with the first jaw 106 and the second jaw 108 in an open, tissue-receiving configuration. The first and second jaws 106, 108 of this embodiment are generally cup-shaped with convex outer surfaces and concave inner surfaces such that, in the closed configuration, an inner tissue-receiving space 109 is formed between the first and second jaws 106, 108. The outer perimeter edges of the first and second jaws 106, 108 are formed as tissue cutting edges 130, 132 configured to mate with one another when in the closed configuration. For example, in this embodiment, the perimeters of the first and second jaws 106, 108 include complimentary serrated edges or teeth such that peaks of the serrations of the first jaw 106 fit within the valleys of the serrations of the second jaw 108, and vice versa. In another embodiment, the distal cutting edges 130, 132 may be straight cutting edges. The first jaw 106 includes a proximal tang 119 with a cylindrical extrude feature 122 (which acts like a pivot pin) sized and shaped to pass through a pivot hole 140 in the clevis 110. The second jaw 108 also includes a proximal tang 126 also defining a cylindrical extrude feature 122 (which acts as a pivot pin) sized and shaped to pass through the pivot hole 140. The cylindrical extrude features 122, 124 pass through the pivot hole 140 in a direction transverse to a central longitudinal axis of the forceps assembly 10. The first and second jaws 106, 108 further include cam pins 123, 125, respectively, extending from an inner surface of the tangs 119, 126 and configured to be inserted into cam slots 152, 154 of the control wire attachment 112, as will be discussed in further detail below. The cam pins 123, 125 are located proximally of the cylindrical extrude features 122 with the cam pin 123 located closer to an upper surface of the first jaw tang 119 and the cam pin 125 located closer to a lower surface of the second jaw tang 126 so that, as the control wire attachment 112 is moved distally, the cam pins 123, 125 glide in opposing directions through the cam slots 152, 154, radially opening the jaws 106, 108.

The clevis 110 is substantially U-shaped and includes a central lumen 134. The clevis 110 includes a pair of arms 136 extending distally from a generally cylindrical proximal portion 138. The central lumen 134 passes through the proximal portion 138 and is sized and shaped to receive the control wire attachment 112 therein. Each arm 136 has a generally curved outer surface and a generally flat inner surface and includes a pivot hole 140 to receive the cylindrical extrude features 122 from the jaws 106, 108 therein. A jaw receiving space 143 is defined between the two arms 136,137 to receive the tangs 119, 126 of the first and second jaws 106, 108, respectively. The pivot pin 124 extends through the pivot pin holes 124 of the arms 136 as well as the mounting holes 122 of the first and second jaws 106, 108 to connect the clevis 110 to the first and second jaws 106, 108 and permit pivotal movement of the first and second jaws 106, 108 relative to one another and the clevis 110. The control wire attachment 112 extends through the clevis lumen 134 and connects to a distal end of the control wire 120 housed within the elongate member 104. These connections couple the distal assembly 100 to the proximal assembly 102 while the control wire 120 and control wire attachment 112 are used to actuate the jaws 106, 108, as described in further detail below.

The control wire attachment 112, as shown in FIGS. 4-5, extends from a proximal end 142 to a distal end 144 and includes a proximal part 146 and a distal part 148. The proximal part 146 is substantially cylindrical and defines a central blind hole 143 open at the proximal end 142. The blind hole 143 is configured to be welded to the distal end of the control wire 120. The distal part 148 includes generally flat lateral surfaces which taper radially inward toward a longitudinal axis of the forceps assembly 10 to a distal spike 150 which assists in anchoring the forceps assembly 10 to target tissue and increasing depth of tissue penetration. The spike 150 also aids in retaining tissue captured within the distal assembly 100 when the forceps assembly 10 is pulled away from the targeted tissue site. The tip of the spike 150 in this embodiment terminates at a small flat square shape or, alternatively, may terminate at a sharp spike. The control wire attachment 112 also includes two cam slots 152, 154 on each of the outer surfaces thereof. Each cam slot 152, 154 is arcuately shaped, as most easily seen in FIG. 5, and couples with the cam pins 123, 125, respectively, of the first and second jaws 106, 108, respectively. As best seen in FIG. 5, the cam slot 152 extends into a first surface of the control wire attachment 112 and is concavely curved from a lower proximal portion of the distal part 148 to an upper distal portion of the distal part 148. The cam slot 154 extends into a second, opposing surface of the control wire attachment 112 and is concavely curved from an upper proximal portion of the distal part 148 to a lower distal portion of the distal part 148. Thus, the cam slots 152, 154 are curved in opposite directions and cross one another with a medial portion of each overlapping to form an open through-hole 151 extending the width (i.e., dimension in a direction perpendicular to the longitudinal axis of the forceps assembly 10 between the outer surfaces of the control wire attachment 112) of the control wire attachment 112. The cam slots 152, 154 are sized and shaped to receive the cam pins 123, 125 therein and allow for radial movement of the first and second jaws 106, 108 about a pivot point as the control wire attachment 112 is moved along the longitudinal axis, L, of the forceps assembly 10. Specifically, as the control wire attachment 112 is moved in a distal direction, the cam pins 123, 125 glide from distal ends of the slots 152, 154 to proximal ends of the slots 152, 154, causing the first and second jaws 106, 108 to move to the open, tissue-receiving configuration, as described in further detail below. Conversely, as the control wire attachment 112 is moved proximally, the cam pins 123,125 glide from proximal ends of the slots 152, 154, to distal ends thereof, causing the first and second jaws 106, 108 to move to the closed configuration.

In an exemplary embodiment, the distal assembly 100 has a rigid portion with a reduced length of, for example, 4.95 mm, when compared to standard forceps end effectors. Specifically, the jaws 106, 108 are reduced in length by approximately 0.3 mm to a rigid length of, for example, 3.48 mm. Furthermore, the clevis 110 is reduced in length by 1.278 mm to a rigid length of, for example, 2.85 mm. The shortening of these components and, thus, the distal assembly 100, allows the distal assembly 100 to more easily pass through acute curvatures within a living body. Furthermore, the reduced rigid length of the distal assembly 100, in combination with the spike 150, reduces the number of bites required to grab the target tissue, resulting. This reduction in the number of bites required to grab the target tissue results in a smaller number of insertions of the distal assembly 100 into the tissue, reducing trauma to the surrounding tissue.

Turning back to FIG. 1, the elongate member 104 is coupled to, and extends proximally from, the clevis 110. The elongate member 104 and clevis 110 may be coupled to one another via any of a variety of methods including, but not limited to, welding, soldering, adhesives, etc. In an exemplary embodiment, the elongate member 104 may be formed of a flexible, closely wound, stainless steel helical coil and may further include a thin covering or coating, such as a layer of polytetrafluroethelene (PTFE) as would be understood by those skilled in the art. The flexible coil 104 may have, for example, a circular, rectangular, or other cross-section. As one skilled in the art would understand, other shapes for the cross-section may be selected depending on the particular application. The PTFE reduces friction between the working channel of the endoscope and the elongate member 104 so that the forceps assembly 10 slides more easily within the endoscope.

The control wire 120, as depicted in FIG. 1, extends from a proximal end 160 to a distal end 162 (shown in FIG. 3) and is sized and shaped to be slidably movable within the elongate member 104. The proximal end 160 of the control wire 120 is inserted into a hypotube 190 and connected to the spool 118 which is movable along the longitudinal axis of the forceps assembly 10 for actuation of the control wire 120. At the distal end 162, the control wire 120 is coupled to the proximal end of the control wire attachment 112, as can be seen in FIG. 3. The control wire 120 and the control wire attachment 112 may be coupled via any suitable means such as, for example, welding, soldering, adhesives, etc. In an exemplary embodiment, the control wire 120 is sufficiently flexible to be passed through a working channel of an endoscope inserted to a target site within the body via, for example, a natural body lumen accessed via a bodily orifice and passed along the body lumen along a tortuous path. The control wire 120 is preferably formed of a material such as stainless steel exhibiting a torsional stiffness sufficient to transmit rotational force to the distal end of thereof. The control wire 120 may have a constant diameter along its length or may vary depending on the application. For example, in one embodiment, the control wire 120 may include a taper towards its distal end to facilitate insertion into the control wire attachment 112. In an embodiment, the control wire 120 may including a PTFE coating to reduce friction between the control wire 120 and the elongate member 104.

FIG. 6 depicts the proximal assembly 102 of the forceps assembly 10, including the handle 114, with the thumb ring 116, and spool 118. As seen in FIG. 6, the elongate member 104, may include a coil 178 and a coil retainer 180. The elongate member 104 extends proximally from the clevis 110 to connect to handle 114. In order to facilitate a wide range of applications and reach targeted anatomical regions of small cross-section, the elongated biopsy forceps assembly 10 may be formed to a length of between 270 cm and 300 cm, and more preferably between 270 cm and 290 cm. The spool 118 is movable along the longitudinal axis of the forceps assembly 10 and is sized and shaped to be grasped by the user. As noted above, the movable control wire 120 connects to the proximal end 140 of the core attachment member 112, depicted in FIG. 3, and extends proximally within, and is configured to slide relative to, the elongate member 104. The proximal end 160 of the control wire 120 couples to the movable spool 118 to permit the user to actuate the forceps assembly 10.

The handle 114 is hollow and defines a lumen 182 which houses the coil retainer 180. Dual fixation flanges 184 on the coil retainer 180 prevent longitudinal movement of the coil retainer 180 within the handle 114. Specifically, the flanges 184 are configured to mate with corresponding grooves 186 formed within the handle 114 perpendicular to the longitudinal axis of the lumen 182. A proximal end 188 of the coil 178 is welded to the coil retainer 180 with a proximal-most face of the coil 178 abutting a distal face of the coil retainer 180. In another embodiment, the coil retainer 180 may be formed integrally with the coil 178. As noted above, the coil retainer flanges 186 prevent the coil retainer 180 from moving longitudinally within the lumen 182 of the handle 114.

Still referring to FIG. 6, the coil retainer 180 houses a hypotube 190 configured to slide within the coil retainer 180. The hypotube 190 is configured to receive the proximal end of the control wire 120 therein. The control wire 120 is secured by a friction fit within an “S” bend 192 at a proximal end of the hypotube 190 and is prevented from movement relative to the hypotube 190. Alternatively, the control wire 120 may be secured to fit within the “S” bend 192 simply be means of the curved geometry of the hypotube 190. Regardless, this fit, either frictional or geometric, provides sufficient coupling of the control wire 120 to the hypotube 190 such that no other means or method of fixation is necessary to transfer movement of the hypotube 190 to the control wire 120. The handle 114 includes an oval-shaped slot 194 that receives the hypotube 190 housing the proximal end of the control wire 120. The movable spool 118 includes an interior surface that slides along the outside of the handle 114 and slot 194. The spool 118 also includes an integral portion received within the slot 194 which has an “S” channel 196 configured to receive the combined hypotube 190 and control wire 120. As noted above with respect to the control wire 120, the fit may be secured simply by means of geometry of the channel 196. Thus, movement of the spool 118 relative to the handle 114 moves the control wire 120 within the coil 178 permit actuation of the distal end effector assembly 100.

Both the clevis 110 and the jaws 106, 108 may be formed of, for example, stainless steel. Alternatively, these components may be formed from aluminum, brass, polymeric materials, nitinol, titanium, or any other suitable biocompatible material. The components may be manufactured through various methods such as, for example, injection molding, precision machining, casting, etc. Other components including the control wire 120, core attachment member 112 and the pins 123, 124, 125, 127, also can be manufactured from stainless steel or any other suitable biocompatible material, such as those described above.

In use, the forceps assembly 10 is maintained in the closed configuration and inserted into the body, e.g., through the working channel of an insertion instrument such as the endoscope 20 which may be, for example, a SpyScope DS™. The PTFE coating of the flexible coil 178 allows the coil 178 to be inserted through the endoscope and into the body with minimal friction while the smaller dimensions of the distal end effector assembly 100 makes delivery through tightly curved tortuous passageways easier because the length of the stiff components of the device (i.e., jaws 104, 106, clevis 110, control wire retainer 112) is reduced in comparison to traditional biopsy forceps. Specifically, in this embodiment, when a SpyScope is inserted into the biliary tract, it forms an acute angle due to the complex anatomy and location of the biliary tract. The reduced rigid length of the distal assembly is 4.95 mm facilitates passage of the assembly 10 through these tight curves, enhancing maneuverability and positioning at a desired location. The coil 178 along with the distal assembly 100 is passed along the tortuous path and, in this exemplary application, positioned in the common bile duct (CBD) 30, as seen in FIG. 7. Once the distal end assembly 100 has been positioned as desired adjacent to the target tissue, the spool 118 is advanced distally, moving the control wire 120 and thus, the control wire 112 attachment distally. This distal movement of the control wire attachment 112 causes the cam pins 123, 125 of jaws 106, 108, respectively, to glide through the cam slots 152, 154, pivoting the first and second jaws 106, 108 about the cylindrical extrude features 122 to the open, tissue receiving configuration, as shown in FIG. 8. As the jaws 106, 108 open and the control wire attachment 112 advances forward, the anchoring spike 150 pierces into and anchors within the target tissue, helping to increased depth to which an obtained sample of the target tissue may extend, as seen in FIG. 8. The first and second jaws 106, 108 are then closed by withdrawing the control wire 120 proximally, causing the cam pins 123, 125 to travel through the cam slots 152, 154 and the first and second jaws 106, 108 to pivot closed about the cylindrical extrude features 122, as depicted in FIGS. 9-10. As the first and second jaws 106, 108 close, the control wire attachment 112 pulls the anchored tissue into the jaws 106, 108 and the cutting edges along the profile of the first and second jaws 106, 108 sever the tissue captured in the tissue receiving space 109 of the first and second jaws 106, 108 from the surrounding tissue, as shown in FIG. 2. 10-12. Once the tissue has been collected within the first and second jaws 106, 108, the forceps assembly 10 is retracted proximally from the endoscope and the tissue may be retrieved from the first and second jaws 106, 108 for diagnosis. Because the first and second jaws 106, 108 anchor into and capture a larger amount of tissue in each bite, the forceps assembly 10 is capable of providing more conclusive tissue samples and a shorter procedure time. However, if more tissue is preferred for the diagnosis, the forceps assembly 10 may be re-inserted through the endoscope for further tissue extraction in the same manner.

FIGS. 13-16 depict particular control wire attachment designs according to further embodiments. The control wire attachments shown in FIGS. 13-16 are substantially the same as control wire attachment 112 depicted in FIGS. 1-12, except as discussed herein. Regarding FIG. 13, a control wire attachment 212, according to an exemplary embodiment, extends from a proximal end 242 to a distal end 244 and has a proximal part 246 and a distal part 248.

However, in this embodiment, the distal end 244 of the wire attachment 212 includes a knuckle spike feature 250. Specifically, the knuckle spike 250 includes a hooked portion 251, which facilitates tissue anchoring. Furthermore, the knuckle spike 250 allows the tissue to be pulled into the first and second jaws 106, 108 as they are closing, thereby further improving the depth to which tissue may be bitten by the first and second jaws 106, 108.

A control wire attachment 312, according to another exemplary embodiment depicted in FIG. 14, extends from a proximal end 342 to a distal end 344 and includes a proximal part 346 and a distal part 348. In this embodiment, the distal part 348 includes a hollow beveled distal tip 350 open at the distal end 344. The beveled distal tip 350 allows the control wire attachment 312 to penetrate deeper into the tissue. The beveled tip 350 also improves maneuverability to a required depth within the target tissue, improving the tissue volume retrieved with each bite.

Another exemplary embodiment of a control wire attachment 412 is shown in FIG. 15. In this embodiment, the control wire attachment 412 includes a plurality of distally pointing spikes 450a-e rather than a single spike 150 as seen in control wire attachment 112. In the exemplary embodiment of the figure there are five spikes 450a-e. However, this number is exemplary only. Any number of spikes 450 may be used depending on the application. As can be seen in the figure, the distal end 444 of the distal part 448 still has a tapered shape so that a medial spike 450c extends the furthest distally with each subsequent spike extending to a more proximal point. The spikes 450a-e provide the control wire attachment 412 with multiple anchoring points and thus, allow for a greater tissue volume in each bite of the first and second jaws 106, 108.

In another exemplary embodiment shown in FIG. 16, a control wire attachment 512 includes serrated distal tapered edges 551 along the spike 550. These serrated edges 551 provide for improved anchoring of the control wire attachment 512 into the tissue during proximal retraction thereof, thus capturing an increased tissue volume per bite.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this disclosure is not limited to the particular embodiment disclosed, but rather modifications are also covered within the scope of the present disclosure as defined by the appended claims.

1-15. (canceled)

16. A biopsy forceps device, comprising:
a tension member extending from a proximal end to a distal end; and
an end effector including first and second jaws movable between an open configuration, in which the jaws are separated from one another to receive target tissue therebetween, and a closed configuration, in which cutting edges of the jaws are moved toward one another to cut a portion of the target tissue from surrounding tissue, the first and second jaws defining a tissue receiving space therebetween to house the cut tissue, the first and second jaws being pivotable relative to one another, the end effector further including a tension member attachment extending from a distal end to a proximal end coupled to the tension member, the distal end of the tension member attachment terminating at a sharp point configured to penetrate the tissue, the tension member attachment being movably coupled to the first and second jaws so that distal movement of the tension member attachment moves the jaws to the open configuration while proximal movement thereof moves the jaws to the closed configuration.

17. The device of claim 16, wherein the first and second jaws include concave inner surfaces defining a substantially hemispherical cup.

18. The device of claim 16, wherein the tension member attachment includes a proximal part and a distal part, the distal part including first and second cam slots, the first and second cam slots being positioned on opposing outer surfaces of the tension member attachment.

19. The device of claim 18, wherein the first and second jaws each include a cam pin, the cam pin of the first jaw being slidably received within the first cam slot while the cam pin of the second jaw is slidably received within the second cam slot so that movement of tension member attachment relative to the cam pins of the first and second jaws pivots the first and second jaw in opposing directions.

20. The device of claim 18, wherein the first and second cam slots are substantially arcuate in shape, the first and second cam slots curving in opposite directions and crossing one another with a medial portion of each cam slot overlapping.

21. The device of claim 18, wherein the proximal part of the tension member attachment includes a blind hole open at a proximal end in which a distal end of the control wire is received.

22. The device of claim 16, wherein the end effector has a length of less than 5 mm.

23. The device of claim 22, wherein the end effector has a length of less than approximately 4.95 mm.

24. The device of claim 16, further including a clevis, the clevis including first and second arms extending distally from a proximal portion, the proximal portion including a central lumen sized and shaped to receive the tension member attachment therethrough, the aims defining a jaw receiving space therebetween for receiving a proximal portion of each of the first and second arms, the first jaw being pivotably coupled to the first arm and the second jaw being pivotably coupled to the second arm.

25. A biopsy forceps system for sampling tissue, comprising:
a proximal assembly including an actuator;
a tension member extending from a distal end to a proximal end coupled to the proximal assembly; and
a distal assembly including first and second jaws movable between an open configuration, in which the jaws are separated from one another to receive target tissue therebetween, and a closed configuration, in which cutting edges of the jaws are moved toward one another to cut the target tissue from surrounding tissue, the first and second jaws defining a tissue receiving space therebetween to house the cut tissue, the first and second jaws being pivotable relative to one another, the distal assembly further including a tension member attachment extending from a distal end to a proximal end coupled to the tension member, the distal end of the tension member attachment terminating at a tissue penetrating point, the tension member attachment movably coupled to the first and second jaws so that distal movement of the tension member attachment pivots the jaws to the open configuration while proximal movement of the tension member attachment pivots the jaws to the closed configuration;
wherein actuation of the proximal assembly causes the tension member attachment to move proximally and distally relative to the first and second jaws.

26. The system of claim 25, wherein the actuator comprises a handle and a spool, the spool being coupled to the control wire and slidable along a longitudinal axis of the handle.

27. The system of claim 25, wherein the tension member attachment includes a proximal part and a distal part, the distal part including first and second cam slots, the first and second cam slots being positioned on opposing outer surfaces of the tension member attachment.

28. The system of claim 27, wherein each of the first and second jaws includes a cam pin, the cam pin of the first jaw being slidably received in the first cam slot while the cam pin of the second jaw is slidably received the second cam slot so that movement of the tension member attachment relative to the first and second jaws pivots the first and second jaws in opposing directions.

29. The system of claim 27, wherein the first and second cam slots are arcuate, curve in opposite directions and cross one another.

30. The system of claim 16, further including a clevis, the clevis including first and second arms extending distally from a proximal portion of the clevis, the proximal portion including a central lumen sized and shaped to receive the tension wire attachment therein, each of the arms defining a jaw receiving space therebetween for receiving a proximal portion of a corresponding one of the first and second arms, the first jaw being pivotably coupled to the first arm and the second jaw being pivotably coupled to the second arm.

31. A method of obtaining a tissue sample, comprising:
inserting a distal portion of a biopsy forceps assembly to a target area within a living body, the distal portion including:
a tension member extending from a proximal end to a distal end; and
an end effector including first and second jaws movable between an open configuration in which the first and second jaws are separated from one another too receive target tissue therebetween, and a closed configuration, in which cutting edges of the first and second jaws are moved toward one another to cut the target tissue away from surrounding tissue, the first and second jaws defining a tissue receiving space therebetween to house the cut tissue,
moving the tension member distally relative to the first and second jaws to move a tension member attachment coupled to the tension member distally so that a point on a distal end of the tension member attachment penetrates the target tissue, the tension member attachment being movably coupled to the first and second jaws so that distal movement of the tension member attachment relative to the first and second jaws pivots the first and second jaws to the open configuration; and
moving the tension member proximally relative to the first and second jaws to pivot the jaws to the closed configuration so that the cutting edges of the first and second jaws sever the target tissue received therebetween from the surrounding tissue.

32. The method of claim 31, further comprising:
inserting the biopsy forceps assembly through the working channel of an endoscope.

33. The method of claim 31, wherein the tension member attachment includes a proximal part and a distal part, the distal part including first and second cam slots, the first and second cam slots being positioned on opposing outer surfaces of the core wire attachment.

34. The system of claim 33, wherein the first and second jaws each include a cam pin, the cam pin of the first jaw being sized and shaped to slidably move within the first cam slot to pivot the first jaw in a first direction while the cam pin of the second jaw is configured slidably move within the second cam slot to pivot the second jaw in a second, opposing direction.

35. The method of claim 31, further comprising:
actuating the distal portion via a proximal assembly coupled to the proximal end of the tension member.
